(19) United States
(12) Patent Application Publication
(10) Pub. No.: US 2013/0084592 A1
(43) Pub. Date: Apr. 4, 2013

(54) METHODS FOR CORRECTING ASSAY MEASUREMENTS

(71) Applicant: Lauren Amy Seiple, Elkton, MD (US)

(72) Inventor: Lauren Amy Seiple, Elkton, MD (US)

(73) Assignee: SIEMENS HEALTHCARE DIAGNOSTICS INC., Deerfield, IL (US)

(21) Appl. No.: 13/252,693

(22) Filed: Oct. 4, 2011

(51) Int. Cl. G01N 21/25; C12Q 1/02

(52) U.S. Cl. 435/29; 436/501

(57) ABSTRACT

Methods are disclosed for correcting an assay measurement for determining a concentration of an analyte in a sample suspected of containing the analyte. An assay signal is measured at a first wavelength corresponding to the analyte in the sample and an assay signal is measured at a second wavelength corresponding to background that is multiplied by a correction factor. An assay signal value is determined by subtracting assay signal at the second wavelength times the correction factor from assay signal at the first wavelength. The assay signal value is related to the amount of the analyte in the sample.

### BACKGROUND

The invention relates to methods for the determination of the concentration of an analyte in a sample suspected of containing the analyte. More particularly, the invention relates to reducing the effect of interfering substances on measurements conducted during the above methods for the determination of the concentration of an analyte in a sample.

In some examples of assays for an analyte, signal measured may also include artifacts that are not attributable to a reaction product of an analyte with one or more reagents for conducting the assay. For example, in some assays for an analyte, a sample is combined in a medium with reagents for determining an analyte and then the sample is irradiated with light to determine the effect that the analyte, if present, has on one or more of the assay reagents. In such assays, a compound is present in the assay medium that responds to irradiation with light by absorbing light and emitting light at a wavelength indicative of the presence of the analyte in the sample. Such assays are referred to as colorimetric assays or photometric assays. Sample or measurement artifacts may cause the amount of light emitted not to be proportional to the amount of analyte in the sample.

To address this problem, the measurement results are read at two wavelengths, one of which corresponds to the expected wavelength of the compound produced by the reaction between the analyte and one or more of the assay reagents and the other of which is at a wavelength outside that of the expected wavelength. Subtraction of the signal measured at the second wavelength from that at the first wavelength provides the amount of signal attributable solely to the presence of the reaction product of the analyte and one or more of the assay reagents. In this manner, it is thought that contributions to the measurement at the first wavelength from artifacts can be reduced or eliminated by subtracting the amount of signal measured at the second wavelength from the amount of signal measured at the first wavelength.

There is a continuing need to develop fast and accurate diagnostic methods to measure a level of an analyte in a sample taken from a patient. The methods should be fully automatable and be accurate even when conducted on samples having various interfering substances. The assay methods should provide an accurate measurement of the amount of the analyte in the sample while minimizing inaccuracies resulting from interfering substances present in the sample.

### SUMMARY

Some examples in accordance with the principles described herein are directed to methods for correcting an assay measurement for determining a concentration of an analyte in a sample suspected of containing the analyte. An assay signal is measured at a first wavelength corresponding to the analyte in the sample and an assay signal is measured at a second wavelength corresponding to background that is multiplied by a correction factor. An assay signal value is determined by subtracting assay signal at the second wavelength times the correction factor from assay signal at the first wavelength. The assay signal value is related to the amount of the analyte in the sample.

Some examples in accordance with the principles described herein are directed to methods for determining one or both of a presence and an amount of lithium ion in a sample suspected of containing lithium ion. A combination of the sample and a binding partner for the lithium ion is provided in a medium. The combination is incubated under conditions for binding of the binding partner to the lithium ion. An assay signal is measured at a first wavelength corresponding to the lithium ion in the sample. An assay signal is measured at a second wavelength corresponding to background and multiplied by a correction factor. The latter is subtracted from the former to determine an assay signal value, which is related to the amount of lithium ion in the sample.

Some examples in accordance with the principles described herein are directed to methods for mitigating lipid interference with a measurement in an assay for a metal ion in a blood sample wherein, in the assay, a signal is read at a first wavelength and a signal is read at a second wavelength. The method comprises determining a correction factor for signal read at the second wavelength and subtracting signal read at the second wavelength times the correction factor from signal read at the first wavelength to obtain a measurement result.

### DETAILED DESCRIPTION OF SPECIFIC EMBODIMENTS

### General Discussion

The present inventor has discovered that an assay method that involves subtraction of signal read at a second wavelength from signal read at a first wavelength does not always accurately determine the amount of an analyte in a sample. Interfering substances in certain samples may provide a disproportionate contribution to signal read at the first wavelength than to signal read at the second wavelength. Subtraction of the signal read at the second wavelength from signal read at the first wavelength does not provide a signal value that accurately represents the amount of analyte in the sample. An interfering substance is one that absorbs at a second wavelength and include, but are not limited to, lipids, proteins, and small molecules, for example. The interfering substances may cause an assay measurement to be too high or too low than that obtained in the absence of such interfering substances.

To address this problem in accordance with the principles described herein, the measurement results are read at two wavelengths, one of which corresponds to the expected wavelength of the compound produced by the reaction between the analyte and one or more of the assay reagents and the other of which is at a wavelength outside of that of the expected wavelength. Subtraction of the signal measured at the second wavelength times a correction factor from that measured at the first wavelength provides a correction to the amount of signal attributable solely to the presence of the reaction product of the analyte and one or more of the assay reagents. The present inventor found that using a correction factor as discussed above results in a more accurate determination of an analyte whether the interfering substances cause the assay measurement at a second wavelength to be too high or too low in the absence of a correction factor.

The principles described herein have application to any assay that involves reading of an assay signal at at least two different wavelengths and subtracting one assay signal from another. Such assays are referred to as photometric assays or colorimetric assays. An assay that involves reading signal at two different wavelengths is referred to as a bichromatic photometric assay. In some examples, the assay comprises adding reagents for determining the concentration of the analyte in the sample to a medium comprising the sample. The reagents comprise at least one binding partner for the analyte. The medium is incubated under conditions for binding of the analyte to the binding partner for the analyte. Furthermore, the methods in accordance with the principles described herein have particular application to automated assay methods.

### Determination of Correction Factor

As mentioned above, examples in accordance with the principles described herein have particular application to photometric/colorimetric assays for analytes where interfering substances impact the accuracy of the measurement of signal by a subtraction method. In some examples, the identification of such assays may be carried out using lipid interference and serum samples by way of illustration and not limitation.

In some examples, the correction factor is determined empirically. An assay in question is employed for samples having known amounts of one or more interfering substances of concern wherein the amount of analyte is the same for all samples (known amount). After incubation of assay reagents with the samples, signal is read at two different wavelengths, one that corresponds to analyte (first wavelength) and one that is a wavelength at which analyte contribution is negligible (second wavelength). Signals at the second wavelength are multiplied by correction factors of incrementally increasing value and the product of that multiplication in each instance is subtracted from a respective signal at the first wavelength to give a signal value for each sample tested. The results are analyzed to determine which correction factor yields the lowest number of samples with a significant (plus or minus a predetermined percentage) bias from the known amount of analyte. This correction factor may then be used in any photometric assay for such an analyte using the same reagents and assay format that were employed in the empirical determination. A different correction factor is determined for an assay wherein one or more of the analyte, the assay format and the assay reagents differ from an assay for which a correction factor has been determined.

In some examples the correction factor is determined mathematically by measuring the change in signal at the first wavelength for an analyte-free sample upon the introduction of increasing concentrations of one or more interfering substances and dividing it by the change in signal at the second wavelength for the same sample upon the introduction of increasing concentrations of one or more interfering substances. This value is used as a starting point in the selection of the appropriate correction factor as described above.

### Empirical Determination of Correction Factor

A series of native lipemic analyte-free patient serum samples are spiked with a known concentration of analyte. The samples are analyzed using the assay of interest and biases from the expected value are determined. In some examples, spiking accuracy is verified using a second methodology refractory to lipid interference. The samples are also analyzed for lipemic index, and analyte recovery versus lipemic index is plotted. The presence of lipid interference indicates that the assay is a candidate for the methods described herein.

Lipid (in the form of a fat emulsion) is spiked into analyte-free serum at increasing concentrations and the samples are analyzed using the assay of interest. Photometric data is collected from the analyzer and endpoint absorbance is plotted versus wavelength for each sample. Any lipid-dependent shifts in absorbance at the primary and secondary wavelengths are observed. A correction factor is determined when a lipid-dependent absorbance shift seen at the secondary wavelength differs in magnitude from that observed at the primary wavelength.

In the method, a serum pool is spiked with a known amount of analyte and is analyzed in replicate using the method with weighting factors of increasing magnitude. Recovery and within-run precision are determined. Any increase in method bias or imprecision correlating with the magnitude of a correction factor is taken into consideration during final selection of a correction factor.

The ability of a correction factor to correct for lipid interference in native human serum samples is then assessed. Lipemic patient samples are spiked with a known concentration of analyte and analyzed using the method of interest and an analyzer. Photometric data is obtained from the analyzer and used to determine method recovery when increasing correction factors are included. The number of samples recovering within ±10% from the expected value using each correction factor is determined. The correction factor that is the most successful at correcting for lipid interference while still demonstrating acceptable recovery and precision performance is chosen for inclusion in the method.

The correction factor is then verified. A subset of the native lipemic samples used during initial interference testing is reanalyzed using the method of interest and the selected correction factor. Percent bias from the expected analyte value is calculated. If the inclusion of the correction factor significantly improves the performance of the assay when measuring analyte in lipemic samples, it is employed in final assay design.

### General Description of Assays for an Analyte

As mentioned above, the principles described herein have application to any assay for an analyte that involves measuring assay signal at at least two different wavelengths. The assays are conducted by combining in an assay medium a sample and reagents for determining the amount of the analyte in the sample. The nature of the reagents is dependent on the particular type of assay to be performed. In general, the assay is a method for the determination of the amount of an analyte in a sample.

The sample to be tested may be non-biological or biological. “Non-biological samples” are those that do not relate to a biological material and include, for example, soil samples, water samples, air samples, samples of other gases and mineral samples. The phrase “biological sample” refers to any biological material such as, for example, body fluid, body tissue, body compounds and culture media. The sample may be a solid, semi-solid or a fluid (a liquid or a gas) from any source. In some embodiments the sample may be a body excretion, a body aspirant, a body excisant or a body extractant. The body is usually that of a mammal and in some embodiments the body is a human body. Body excretions are those substances that are excreted from a body (although they also may be obtained by excision or extraction) such as, for example, urine, feces, stool, vaginal mucus, semen, tears, breath, sweat, blister fluid and inflammatory exudates. Body excisants are those materials that are excised from a body such as, for example, skin, hair and tissue samples including biopsies from organs and other body parts. Body aspirants are those materials that are aspirated from a body such as, for example, mucus, saliva and sputum. Body extractants are those materials that are extracted from a body such as, for example, whole blood, plasma, serum, spinal fluid, cerebral spinal fluid, lymphatic fluid, synovial fluid and peritoneal fluid. In some examples the sample is whole blood, plasma or serum.

The analyte is a substance of interest or the compound or composition to be detected and/or quantified. Analytes include, by way of illustration and not limitation, therapeutic drugs, drugs of abuse, metabolites, proteins (such as, for example, enzymes, plasma proteins, and antibodies), polysaccharides, polysaccharide-protein combinations, pollutants, pesticides, volatile organic compounds, semi-volatile organic compounds, non-volatile organic compounds, toxins, and nucleic acids (DNA, RNA), for example. Therapeutic drug analytes include, but are not limited to, metal ions, small organic compounds (molecular weight less than about 2500), proteins, nucleic acids, polynucleotides, and steroids, for example. Therapeutic metal ions include, for example, lithium ion (treatment of depression), magnesium ion (treatment of preeclampsia), and calcium ion (treatment of preeclampsia).

The assay may be a non-immunoassay or an immunoassay. Various assay methods are discussed below by way of illustration and not limitation. In some examples, homogeneous immunoassays may be employed; such assays may also be referred to as essentially partition-free immunoassays. The present methods have application to fully automated homogeneous assays.

In many embodiments the reagents comprise at least one binding partner for an analyte. As used herein, the phrase “binding partner for an analyte” refers to a compound that reacts with the analyte to form a covalent, non-covalent or ionic bond and in that sense binds to the other entity; the term also refers to compounds that react with an analyte and convert the analyte into a chromophore or otherwise alter or change the chromophoric nature of an analyte. The binding partner may be, by way of illustration and not limitation, a chromophore (non-immunoassay), an antibody or antigen for the analyte (immunoassay), nucleic acid, or an enzyme or substrate for the analyte, for example. The term “chromophore” refers to a light absorbing compound. Chromophores include, but are not limited to, chromoionophores (chromogenic ionophore, binds to ions), chromogenic substrates (binds to enzymes), and chromogenic cofactors (produced by an enzymatic reaction), for example.

The phrase “antibody for the analyte” refers to an antibody that binds specifically to an analyte (and in some example to closely related structural analogs of the analyte such as metabolites of the analyte) and does not bind to any significant degree to other substances that would distort the analysis for the analyte. Accordingly, specific binding involves the specific recognition of one of two different molecules for the other compared to substantially less recognition of other molecules. On the other hand, non-specific binding involves non-covalent binding between molecules that is relatively independent of specific surface structures. Non-specific binding may result from several factors including hydrophobic interactions between molecules. In many embodiments of assays, preferred binding partners are antibodies and the assays are referred to as immunoassays.

One general group of non-immunoassays, to which the present principles have application, is assays involving chromoionophores used in the detection of metal ions. Another group of non-immunoassays, to which the present principles have application, is assays for the determination of enzymes where a chromogenic substrate or cofactor is employed. Another group of non-immunoassays, to which the present principles have application, is assays involving the use of a reactant that alters the light absorbing properties of an analyte.

The principles in accordance with the present disclosure also have application to immunoassays wherein a chromophore is formed or altered during the assay. In some examples, the binding of an antibody for the analyte to the analyte results in the formation or alteration of a chromophore such as, for example, a chromogenic substrate or a chromoionophore. Alteration of a chromophore refers to the change in a chromophore so that the chromophore emits light at a different wavelength than the unchanged chromophore. The chromophore may be a label used in the assay, the analyte itself, or a cofactor, for example.

Other reagents are included in the assay medium depending on the nature of the assay to be conducted. Immunoassays may involve labeled or non-labeled reagents. Labeled immunoassays include enzyme immunoassays, fluorescence polarization immunoassays, induced luminescence assays, fluorescent oxygen channeling assays, by way of illustration and not limitation. The assays can be performed either without separation (homogeneous) or with separation (heterogeneous) of any of the assay components or products. The label or other members of an assay reagent system can be bound to a support, which can have any of a number of shapes, such as particle (including bead), film, membrane, tube, well, strip, rod, planar surfaces such as, e.g., plate, paper, etc., fiber, and the like.

The assays discussed above are normally carried out in an aqueous medium at a variable pH, generally that which provides optimum assay sensitivity. The pH for the assay medium will usually be in the range of about 4 to about 13, or in the range of about 5 to about 12, or in the range of about 6.5 to about 9.5. The pH will usually be a compromise between optimum binding of a binding partner with an analyte and the pH optimum for other reagents of the assay, for example.

If a buffer is employed to achieve the desired pH and maintain the pH during the determination, illustrative buffers include borate, phosphate, carbonate, tris, barbital and the like. The particular buffer employed is not critical, but in an individual assay one or another buffer may be preferred. Various ancillary materials may be employed in the above methods. For example, in addition to buffers the medium may comprise stabilizers for the medium and for the reagents employed. In some embodiments, in addition to these additives, proteins may be included, such as albumins; quaternary ammonium salts; polyanions such as dextran sulfate; binding enhancers, or the like. All of the above materials are present in a concentration or amount sufficient to achieve the desired effect or function.

One or more incubation periods may be applied to the medium at one or more intervals including any intervals between additions of various reagents mentioned above. The medium is usually incubated at a temperature and for a time sufficient for binding of various components of the reagents to occur. Moderate temperatures are normally employed for carrying out an assay and usually constant temperature, preferably, room temperature, during the period of the measurement. Incubation temperatures normally range from about 5° to about 99° C., or from about 15° C. to about 70° C., or about 20° C. to about 45° C., for example. The time period for the incubation is about 0.2 seconds to about 24 hours, or about 1 second to about 6 hours, or about 2 seconds to about 1 hour, or about 1 to about 15 minutes, for example. The time period depends on the temperature of the medium and the rate of binding of the various reagents. Temperatures during measurements will generally range from about 10 to about 50° C., or from about 15 to about 40° C.

The concentration of analyte that may be assayed generally varies from about 10⁻² to about 10⁻¹⁷ M, or from about 10⁻⁶ to about 10⁻¹⁴ M. Considerations, such as whether the assay is qualitative, semi-quantitative or quantitative (relative to the amount of erythrocytophilic drug analyte present in the sample), the particular detection technique and the concentration of the analyte normally determine the concentrations of the various reagents.

The concentrations of the various reagents in the assay medium will generally be determined by the concentration range of interest of the analyte, the nature of the assay, and the nature of the assay reagents, for example. However, the final concentration of each of the reagents is normally determined empirically to optimize the sensitivity of the assay over the range of interest. That is, a variation in concentration of analyte that is of significance should provide an accurately measurable signal difference.

While the order of addition may be varied widely, there will be certain preferences depending on the nature of the assay. The simplest order of addition is to add all the materials simultaneously and determine the effect that the assay medium has on the signal as in a homogeneous assay. Alternatively, the reagents can be combined sequentially. Optionally, an incubation step may be involved subsequent to each addition as discussed above.

### Measurement Step

The measurement is carried out respectively for each assay medium following the incubation of the assay medium in accordance with the particular assay employed. The phrase “measuring the amount of an analyte” refers to the quantitative, semi-quantitative and qualitative determination of the analyte. Methods that are quantitative, semi-quantitative and qualitative, as well as all other methods for determining the analyte, are considered to be methods of measuring the amount of the analyte. For example, a method, which merely detects the presence or absence of the analyte in a sample suspected of containing the analyte, is considered to be included within the scope of the present disclosure. The terms “detecting” and “determining,” as well as other common synonyms for measuring, are contemplated within the scope of the present disclosure.

As indicated above, signal is detected from the assay medium at two different wavelengths. The amount of signal attributable solely to the presence of the analyte in the sample is determined by subtracting the measurements at two different wavelengths wherein a correction factor is employed prior to subtraction in accordance with the principles described herein. The amount of the signal is related to the amount of the analyte in the sample. Prior to the measurement, the medium is irradiated with light. The examination for amount of the signal also includes the detection of the signal, which is generally merely a step in which the signal is read. The signal is normally read using an instrument such as, for example, a spectrophotometer, fluorometer, absorption spectrometer, luminometer, or chemiluminometer, for example. The amount of signal detected is related to the amount of the analyte present in a sample. Temperatures during measurements generally range from about 10° to about 70° C., or from about 20° to about 45° C., or about 20° to about 25° C., for example. In one approach standard curves are formed using known concentrations of the analytes to be screened. As discussed herein, calibrators and other controls may also be used.

### Example of an Assay for Determination of Lithium Ion

In an example in accordance with the principles described herein, by way of illustration and not limitation, the analyte is lithium and the assay employs a diazocryptand chromoionophore that reacts with the lithium ion. The sample to be analyzed is one that is suspected of containing lithium ion. In this example, the sample is serum. Some of the samples to be analyzed contain one or more lipid interfering substances, which include, but are not limited to, triglycerides, lipoproteins and chylomicrons. The sample is combined in an aqueous buffered medium (pH≧12) with the chromoionophore. The medium is then incubated at a temperature of about 25° C. to about 40° C. for a period of about 10 seconds (sec) to about 2 minutes and then is irradiated with light across the visible spectrum. Signal is read using a photometer at two different wavelengths, 510 nm (corresponding to the lithium ion-diazocryptand chromoionophore complex if lithium ion is present) and 700 nm (a wavelength where little if any contribution to the signal corresponds to the lithium ion-diazocryptand chromoionophore complex if lithium ion is present). The amount of signal at the 700 nm wavelength is multiplied by a correction factor of 1.75, which was previously determined empirically for samples having known concentrations of lipid interfering substances. The product of the multiplication is subtracted from the amount of signal read at the 510 nm wavelength to give a value of signal, which is then related to the amount of lithium ion in the sample.

### Kits for Conducting Assays

The reagents for conducting a particular assay may be present in a kit useful for conveniently performing an assay for the determination of an analyte. In one example, a kit comprises in packaged combination reagents for analyzing for an analyte, the nature of which depend upon the particular assay format. The reagents may include, for example, a binding partner for the analyte. The reagents may each be in separate containers or various reagents can be combined in one or more containers depending on the cross-reactivity and stability of the reagents. The kit can further include other separately packaged reagents for conducting an assay such as additional binding members and ancillary reagents.

The relative amounts of the various reagents in the kits can be varied widely to provide for concentrations of the reagents that substantially optimize the reactions that need to occur during the present method and further to optimize substantially the sensitivity of the assay. Under appropriate circumstances one or more of the reagents in the kit can be provided as a dry powder, usually lyophilized, including excipients, which on dissolution will provide for a reagent solution having the appropriate concentrations for performing a method or assay. The kit can further include a written description of a method in accordance with the present embodiments as described above.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The following examples further describe the specific embodiments of the invention by way of illustration and not limitation and are intended to describe and not to limit the scope of the invention. Parts and percentages disclosed herein are by volume unless otherwise indicated.

### EXAMPLES

The diazocryptand chromoionophore dye (Dye) was manufactured by Siemens Healthcare Diagnostics Inc., Elkhart Ind. (see U.S. Pat. No. 5,187,103, the relevant portions thereof being incorporated herein by reference). All other chemicals were purchased from the Sigma-Aldrich Company, St. Louis Mo. (2(3)-tert-butyl-4-hydroxyanisole, diethylene glycol monoethyl ether, PROCLIN® 300 preservative), VWR International, Radnor Pa. (TRITON® X-100 surfactant, hydrochloric acid, potassium hydroxide) or Fisher Scientific, Pittsburgh Pa. (potassium hydroxide pellet). Testing was carried out using the DIMENSION® VISTA® Intelligent Lab System analyzer from Siemens Healthcare Diagnostics Inc., Newark Del. The instrument is employed using a lithium chromogenic assay (the lithium assay) substantially as described in U.S. Pat. No. 5,187,103, the relevant portions thereof being incorporated herein by reference. This assay employs a diazocryptand chromoionophore dye—lithium binding reaction depicted below.

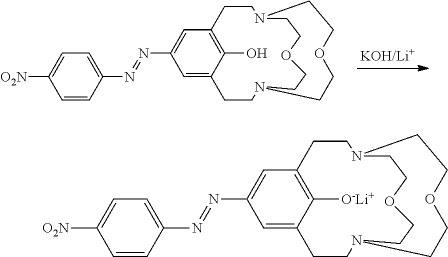

For the assay, sample volume was 2 μL. Reagent Composition is as set forth below in Table 1.

TABLE 1

| Reagent | Form | Ingredient | Concentration |
|---|---|---|---|
| Dye | Liquid | Dye | 8.05 × 10⁻⁴M |
| reagent | | Diethylene glycol monoethyl ether | 5% (v/v) |
| | | 2(3)-tert-Butyl-4-hydroxyanisole | 0.01% (w/v) |
| | | Surfactant | |
| | | Preservative | |
| Alkaline | Liquid | KOH | 1M |
| reagent | | Diethylene glycol monoethyl ether | 5% (v/v) |
| | | Surfactant | |

Alkaline Reagent Volume was 86 μL; Dye Reagent Volume was 43 μL; sample incubation time was 29 sec.; and the temperature was 37° C. All reagent and sample additions were neat. Alkaline reagent was preincubated with dye reagent and a reagent blank measurement was read prior to sample addition. This reagent blank absorbance was subtracted from the endpoint absorbance values in the final calculation.

### Identification of Lipid Interference

A series of native lipemic lithium-free patient serum samples were spiked with a known concentration of lithium (in the form of lithium carbonate). The samples were analyzed using the lithium assay and biases from the expected value were determined. The samples were also analyzed for lipemic index using the DIMENSION® VISTA® HIL (Siemens Healthcare Diagnostics Inc., Newark Del.) feature and lithium recovery versus lipemic index was plotted. (Spiking accuracy was verified using a second methodology (DIMENSION® LI, Siemens Healthcare Diagnostics Inc., Newark Del.) refractory to lipid interference.)

### Primary and Secondary Wavelength Confirmation

A series of non-lipemic samples of increasing lithium concentration were obtained and analyzed using the lithium assay. Photometric data was collected from the analyzer and endpoint absorbance was plotted versus wavelength for each sample. Lithium-dependent shifts in absorbance were seen at several wavelengths, with the greatest shift observed at 510 nm. Low background absorbance was seen at 700 nm, with no lithium-dependent change in signal. This confirmed 510 nm and 700 nm as the appropriate primary and secondary wavelengths, respectively, for the bichromatic endpoint read.

### Determination of the Impact of Lipid on Reaction Absorbance

Lipid (in the form of INTRALIPID® 20% fat emulsion, Fresenius Kabi, Bad Homburg, Germany) was spiked into lithium-free serum at increasing concentrations and the samples were analyzed using the aforementioned lithium assay. Photometric data was collected from the analyzer and endpoint absorbance was plotted versus wavelength for each sample. Lipid-dependent shifts of varying magnitudes in absorbance were observed at nearly every wavelength collected, including 510 nm and 700 nm.

### Determination of Correction Factor

Because the lipid-dependent absorbance shift seen at 700 nm was lesser in magnitude than that observed at 510 nm, the addition of a 700 nm correction factor was pursued. However, since the addition of a correction factor at 700 nm will increase the background noise of the lithium method, the impact of such a factor on method recovery and precision was determined. A serum pool spiked with a known amount of lithium (in the form of lithium carbonate) was analyzed in replicate (N=6) using the lithium method with correction factors of increasing magnitude and recovery and within-run precision were determined. Method bias and imprecision did increase with weighting factor magnitude. This information was taken into consideration during final weighting factor selection.

Fifty-three lipemic patient samples were spiked with a known concentration of lithium (in the form of lithium carbonate) and analyzed using the lithium assay. Photometric data was obtained from the analyzer and used to determine method recovery upon the inclusion of a 700 nm correction factor. Factors were tested in increments of 0.25 over a range of 1.00 (no weighting) to 2.50. The number of samples recovering within ±10% from the expected value using each weighting factor was determined. A factor of 1.75 was the most successful at correcting for lipid interference while still demonstrating acceptable recovery and precision performance. The results are summarized in Table 2.

TABLE 2

| | 700 nm Correction Factor | | | | | |
|---|---|---|---|---|---|---|
| | 1 | 1.5 | 1.75 | 2 | 2.25 | 2.5 |
| N = 6 Serum | | | | | | |
| Mean | 0.60 | 0.61 | 0.61 | 0.61 | 0.62 | 0.62 |
| S.D. | 0.004 | 0.007 | 0.010 | 0.012 | 0.014 | 0.017 |
| % CV | 0.61 | 1.21 | 1.57 | 1.94 | 2.30 | 2.66 |
| Lithium | | | | | | |
| Recovery of | | | | | | |
| Lipemic | | | | | | |
| Samples | | | | | | |
| Low (>10%) | 22 | 8 | 6 | 12 | 15 | 17 |
| Bias | | | | | | |
| Expected | 16 | 40 | 46 | 40 | 37 | 33 |
| High (>10%) | 15 | 5 | 1 | 1 | 1 | 3 |
| Bias | | | | | | |
| Total | 53 | 53 | 53 | 53 | 53 | 53 |

### Verification of Correction Factor

A subset of the native lipemic samples used during initial interference testing were reanalyzed using the lithium assay and using the selected 700 nm correction factor of 1.75. Percent bias from the expected value was calculated. The inclusion of the 1.75 correction factor significantly improved the performance of the lithium assay when measuring lithium analyte in lipemic samples. The results are summarized in Table 3 below.

TABLE 3

| | Without Correction Factor | | With Correction Factor | |
|---|---|---|---|---|
| | Lithium | | Lithium | |
| Sample | (mmol/L) | % Bias* | (mmol/L) | % Bias* |
| 1 | 0.62 | 3 | 0.60 | 0 |
| 2 | 0.57 | −5 | 0.61 | 2 |
| 3 | 0.57 | −5 | 0.61 | 2 |
| 4 | 0.56 | −7 | 0.61 | 2 |
| 5 | 0.54 | −10 | 0.6 | 0 |
| 6 | 0.55 | −8 | 0.62 | 3 |
| 7 | 0.57 | −5 | 0.59 | −2 |
| 8 | 0.63 | 5 | 0.64 | 7 |
| 9 | 0.58 | −3 | 0.60 | 0 |
| 10 | 0.54 | −10 | 0.58 | −3 |
| 11 | 0.47 | −22 | 0.56 | −7 |
| 12 | 0.53 | −12 | 0.56 | −7 |
| 13 | 0.54 | −10 | 0.55 | −8 |
| 14 | 0.54 | −10 | 0.60 | 0 |
| 15 | 0.52 | −13 | 0.58 | −3 |
| 16 | 0.50 | −17 | 0.56 | −7 |
| 17 | 0.65 | 8 | 0.58 | −3 |
| 18 | 0.73 | 22 | 0.58 | −3 |
| 19 | 0.62 | 3 | 0.58 | −3 |
| 20 | 0.64 | 7 | 0.63 | 5 |
| 21 | 0.70 | 17 | 0.56 | −7 |
| 22 | 0.90 | 50 | 0.54 | −10 |
| 23 | 0.70 | 17 | 0.61 | 2 |
| 24 | 0.99 | 65 | 0.50 | −17 |
| 25 | 1.41 | 135 | 0.60 | 0 |

*Percent bias based upon an expected value of 0.60 mmol/L

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for correcting an assay measurement for determining a concentration of an analyte in a sample suspected of containing the analyte, the method comprising:
(a) measuring assay signal at a first wavelength corresponding to the analyte in the sample,
(b) measuring assay signal at a second wavelength corresponding to background and multiplying by a correction factor,
(c) subtracting (b) from (a) to determine an assay signal value, and
(d) relating the assay signal value to the concentration of analyte in the sample.

2. The method according to claim 1 wherein the measuring of (a) and (b) are carried out for an assay conducted on the sample, wherein the assay comprises:
(i) adding reagents for determining the concentration of the analyte in the sample to a medium comprising the sample wherein the reagents comprise at least one binding partner for the analyte, and
(ii) incubating the medium under conditions for binding of the analyte to the binding partner for the analyte.

3. The method according to claim 2 wherein the assay is selected from the group consisting of colorimetric assays and photometric assays.

4. The method according to claim 1 wherein the analyte is a metal ion.

5. The method according to claim 1 wherein the analyte is lithium ion.

6. The method according to claim 1 wherein the sample is a body excretion, body aspirant, body excisant or body extractant.

7. A method for determining one or both of a presence and an amount of lithium ion in a sample suspected of containing lithium ion, the method comprising:
(a) providing in combination in a medium the sample and a binding partner for the lithium ion,
(b) incubating the combination under conditions for binding of the binding partner to the lithium ion,
(c) measuring an assay signal at a first wavelength corresponding to the lithium ion in the sample,
(d) measuring assay signal at a second wavelength corresponding to background and multiplying by a correction factor,
(c) subtracting (d) from (c) to determine an assay signal value, and
(d) relating the assay signal value to the concentration of lithium ion in the sample.

8. The method according to claim 7 wherein the binding partner for the lithium ion is a chromogenic ionophore.

9. The method according to claim 7 wherein the binding partner for the lithium ion is a diazocryptand.

10. The method according to claim 7 wherein the first wavelength is 510 nm and the second wavelength is 700 nm.

11. The method according to claim 7 wherein the correction factor is determined empirically.

12. The method according to claim 7 wherein the sample is a body excretion, body aspirant, body excisant or body extractant.

13. The method according to claim 7 wherein the sample is whole blood, plasma, or serum.

14. The method according to claim 13 wherein the sample comprises one or more lipids.

15. A method for mitigating lipid interference with a measurement in an assay for a metal ion in a blood sample, wherein a signal is read at a first wavelength and at a second wavelength, the method comprising:
(a) determining a correction factor for signal read at the second wavelength, and
(b) subtracting signal read at the second wavelength times the correction factor from signal read at the first wavelength to obtain a measurement result.

16. The method according to claim 15 further comprising relating the measurement result to one or both of a presence and an amount of the metal ion in the blood sample.

17. The method according to claim 15 wherein the analyte is lithium ion.

18. The method according to claim 15 wherein the sample is blood serum or plasma.

19. The method according to claim 15 wherein the first wavelength is 510 nm and the second wavelength is 700 nm.

20. The method according to claim 15 wherein the correction factor is determined empirically.
